(19) United States
(12) Patent Application Publication
(10) Pub. No.: US 2017/0304006 A1
(43) Pub. Date: Oct. 26, 2017

(54) Method for Determining the Structure of a Medical Implant for Replacing Removed Tissue

(71) Applicant: Brainlab AG, Munich (DE)

(72) Inventor: Bálint VARKUTI, Munich (DE)

(21) Appl. No.: 15/517,034

(22) PCT Filed: Dec. 1, 2014
(86) PCT No.: PCT/EP2014/076109

§ 371 Date: Apr. 5, 2017

(51) Int. Cl. A61B 34/10; A61F 2/02; G06F 19/00; A61B 34/20; A61B 90/00

(52) U.S. Cl. CPC A61B 34/10; A61B 34/20; A61F 2/02; G06F 19/3437; A61B 2090/397; A61B 2034/108; A61B 2090/3954; A61B 2090/3937; A61B 2090/3925; A61B 2034/2072; A61F 2240/002; A61F 2250/0097; A61F 2250/0098; A61B 2090/3966

(57) ABSTRACT

A data processing method performed by a computer (2) for determining the structure of a medical implant (12; 18; 20; 22) which is to replace removed tissue in a patient's body, comprising the steps of:—acquiring a 3D dataset which represents remaining tissue (10; 17) which at least partly surrounded the removed tissue before the latter was removed;—determining the required contour of the implant (12; 18; 20; 22) from the 3D dataset;—simulating forces exerted by the remaining tissue (10; 17) on the contour of the implant (12; 18; 20;22); and—determining a structure dataset which represents the structure of the implant (12; 18; 20; 22) such that the implant (12; 18; 20; 22) has the required contour and can absorb the simulated forces.

The present invention relates to a customized medical implant and a method for determining the structure of the medical implant which is to replace removed tissue in a patient's body, and to a corresponding computer program and system.

If tissue is removed from a patient's body, for example by surgery, then a volume—which may also be referred to here as a cavity—arises at the location where the removed tissue was previously situated. If this volume remains empty or fills with matter such as a body fluid, the remaining tissue which surrounds the volume may shift into the volume, which can result in damage to the remaining tissue. The inventor has found that this can be avoided by filling the volume with a suitable medical implant, thus replacing the removed tissue.

The removed tissue is for example a part of the brain, such as a tumour and/or brain tissue which is for example removed by cranial resection. If the resulting cavity remains empty or is filled with cerebrospinal fluid, sudden movements of the head can for example cause fibres or other remaining brain tissue to rupture. Similar problems arise in other kinds of resection, such as for example the complete or partial removal of an organ.

The present invention deals with the problem of determining the structure of a medical implant which is to replace the removed tissue in the patient's body. In this document, the term “determine” means that the structure of the medical implant is defined by calculation from relevant determined values, and the term “structure” with regard to the medical implant preferably means the (three-dimensional) shape of the implant, including one or preferably both of the outer shape of the implant and the inner structure of the implant. The outer shape of the implant is preferably determined so as to completely or partly fill the volume surrounded by the remaining tissue. The determined structure of the implant is represented by a structure dataset. In this document, the term “configure”, as also the term “determine”, typically means a non-physical act such as generating information.

The method for determining the structure of a medical implant which is to replace removed tissue in a patient's body comprises the step of acquiring a 3D dataset which represents remaining tissue which at least partly surrounded the removed tissue before the latter was removed. The remaining tissue thus defines the volume to be filled by the medical implant.

The method also comprises the step of determining the required contour of the implant from the 3D dataset. The required contour of the implant is an outer shape of the implant which preferably matches the shape of the removed tissue. However, the contour of the implant need not necessarily match the shape of the cavity completely. It may for example comprise one or more recesses, such that the surrounding tissue is not supported over the whole boundary surface between the removed tissue and the remaining tissue but rather only over one or more parts of the same, such as for example at least 50%, at least 75% or at least 90% of the boundary surface.

The method also comprises the step of simulating forces exerted by the remaining tissue on the contour of the implant. Forces exerted by the remaining tissue are for example caused by gravity or other accelerations which act on the patient's body, such as accelerations caused by the natural movement of the patient or by accidents involving the patient. In this step, the maximum forces which may be assumed are preferably simulated, for example by taking into account the maximum accelerations which may be assumed. It is also for example possible to divide the contour of the implant into a plurality of areas and calculate an average force for each of the areas.

The method also involves the step of determining a structure dataset which represents the structure of the implant such that the implant, for example an implant which is manufactured according to the structure dataset, has the determined contour and can absorb the simulated forces. In this document, the expression “absorb” can mean that the simulated forces are completely absorbed, such that the simulated forces do not cause any deformation of the implant, but can also mean that the implant can be deformed by the simulated forces, such that the implant for example exhibits the same rigidity as the removed tissue. Where properties of the implant are described in this document, this typically means that the structure dataset represents the structure of an implant which has the corresponding properties.

The configured structure dataset can then be applied when producing the implant, for example by using a 3D printer, in particular (but not necessarily) in an intra-operational environment. The result is a customised implant which prevents damage to the remaining tissue.

The implant can be a solid body, which means that the whole contour of the implant is filled with the material or materials from which the implant is made. Preferably, however, the implant is partly or completely hollow, thus reducing the weight of the implant and the amount of material required.

If the implant is a partly or completely hollow body, some or all of the contour of the implant is formed by a wall of the implant. In one embodiment of the method in accordance with the invention, the step of configuring the structure of the implant involves configuring the wall thickness of the implant. The wall thickness is preferably configured such that the implant can absorb the simulated forces. Similar to the above description, the wall thickness can be configured such that the implant exhibits a desired rigidity, such as for example the rigidity of the removed tissue.

In one embodiment, the step of determining the structure dataset involves providing the structure with at least one stiffener. In a hollow implant, the simulated forces might for example necessitate a large wall thickness for the implant. This wall thickness could be reduced by providing one or more stiffeners within the implant. A stiffener can for example exhibit the shape of a beam or a plurality of interacting beams.

The 3D dataset is for example a medical imaging dataset. A medical imaging dataset is obtained using a suitable imaging modality such as CT (computed tomography), MRI (magnetic resonance imaging), x-ray or any other suitable medical imaging technique. The medical imaging dataset is preferably generated once the removed tissue has been removed, such that it represents the actual state of the remaining tissue.

In one embodiment, the step of determining the required contour of the implant from the 3D dataset involves determining the shape of a cavity formed by the remaining tissue in the 3D dataset and using the shape of the cavity as the contour of the implant. In this embodiment, the contour of the implant is derived directly from the 3D dataset. This is for example advantageous if the shape of the remaining tissue is identical before and after removal of the removed tissue.

In another embodiment, the step of determining the required contour of the implant from the 3D dataset involves matching the 3D dataset to an initial 3D dataset which represents at least parts of the remaining tissue before the removed tissue was removed, in order to determine the shape of a cavity formed by the remaining tissue in the matched 3D dataset, and using said shape of the cavity as the contour of the implant. The initial 3D dataset preferably represents all of the remaining tissue. The initial 3D dataset also preferably represents both the remaining tissue and the removed tissue before it was removed.

This embodiment is particularly advantageous if the structure of the remaining tissue in the 3D dataset differs from the structure of the remaining tissue before the removed tissue was removed. The remaining tissue may for example have moved into the cavity due to gravity.

In the matching step described above, the 3D dataset and in particular the remaining tissue in the 3D dataset is matched to the pre-operative state, i.e. before the removed tissue was removed, such that the shape of the cavity in the matched 3D dataset matches the shape of the removed tissue. In the example of a cranial resection, this compensates for any brain shift due to the creation of the cavity and/or the burr hole required for performing the cranial resection.

The initial 3D dataset is for example a medical imaging dataset. This medical imaging dataset is preferably acquired pre-operationally, i.e. before the removed tissue is removed. The initial 3D dataset can also be a matched atlas. A matched atlas is an atlas of a part of a body which is matched to the situation of the particular patient.

In one embodiment, the step of simulating forces exerted by the remaining tissue involves segmenting the remaining tissue on the basis of an atlas and simulating the movement of the segmented remaining tissue. The atlas is preferably matched to a patient's state before the removed tissue was removed. The atlas is for example matched to the initial 3D dataset. If the initial 3D dataset is a matched atlas, then this matched atlas can be used for segmenting the remaining tissue. The atlas could also be matched to the 3D dataset.

Different types of tissue exhibit different properties in terms of for example rigidity and therefore acceleration response. If the remaining tissue is segmented, for example into a plurality of tissue types, then the properties of the respective tissue types can be taken into consideration when simulating the forces.

In one embodiment, the step of determining the structure dataset involves arranging the structure of the implant so as to form at least one marker which is to be detected by a medical navigation system. The structure of the implant can for example form at least one marker sphere, which is a spherical body which can be detected and located by a stereoscopic camera. Another example of a marker is a particular surface design, such as an embossed predetermined shape or a section of the surface of the implant which exhibits particular optical properties, such as for example its colour or reflectivity.

The structure of the implant preferably forms three or more markers, such that the position of the implant, i.e. its spatial location in up to three dimensions and/or alignment in up to three dimensions, can be determined.

The implant can preferably be tracked and/or navigated using the medical navigation system in order to properly position the implant relative to the remaining tissue.

In one embodiment, the step of determining the structure dataset involves providing the structure of the implant with a socket for a marker which is to be detected by a medical navigation system. If a marker, preferably a marker device comprising at least three markers, is attached to the socket, the implant can be tracked and/or navigated using the medical navigation system, for example in order to position the implant relative to the remaining tissue. The position of the socket relative to the implant is preferably known to the medical navigation system.

In one embodiment, the step of determining the structure dataset involves providing the structure of the implant with at least one port and/or with at least one conduit for a medical liquid. A port can for example connect two sides of the implant, preferably two opposing sides of the implant, such that an instrument can be navigated through the port within the implant. Such an instrument can for example be a needle, a cannula, a camera or any other desired instrument. Providing the implant with a port therefore allows the remaining tissue to be accessed through the implant.

If the structure of the implant is provided with a conduit for a medical liquid, the liquid can be easily and accurately administered to the remaining tissue. A conduit can comprise a complex system of conduits, such as for example one inlet and a plurality of outlets, for the medical liquid.

In one embodiment, the method also comprises the steps of producing the medical implant such that it exhibits the structure as defined by the structure dataset, and verifying the contour of the produced implant. As described above, the implant can for example be produced using a 3D printer. The step of verifying the contour preferably involves detecting the contour of the produced implant and comparing the detected contour with the planned contour of the implant. The contour of the produced implant can for example be detected by imaging the produced implant, for example using a medical imaging modality or a 3D scanner, or by sampling the surface or at least predetermined points on the surface using a (tracked) pointer or other tools such as Z-Touch® or Softtouch® provided by the Applicant.

The present invention also relates to a computer program which, when running on a computer, causes the computer to perform the method as described above and/or to a program storage medium on which said program is stored, in particular in a non-transitory form.

The present invention also relates to a system for determining the structure of a medical implant which is to replace removed tissue in a patient's body, comprising a computer on which the aforementioned program is stored and/or run.

Matching is the process of transforming different sets of data into one co-ordinate system. The data can be multiple photographs and/or data from different sensors, different times or different viewpoints. Matching is used in computer visualisation, medical imaging and in compiling and analysing images and data from satellites. Matching is necessary in order to be able to compare or integrate the data obtained from these different measurements and is for example achieved by image fusion.

Image fusion can be elastic image fusion or rigid image fusion. In the case of rigid image fusion, the relative position between the pixels of a 2D image and/or voxels of a 3D image is fixed, while in the case of elastic image fusion, the relative positions are allowed to change.

Elastic fusion transformations (for example, elastic image fusion transformations) are in particular designed to enable a seamless transition from one dataset (for example a first dataset such as for example a first image) to another dataset (for example a second dataset such as for example a second image). The transformation is in particular designed such that one of the first and second datasets (images) is deformed, in particular in such a way that corresponding structures (in particular, corresponding image elements) are arranged at the same position as in the other of the first and second images. The deformed (transformed) image which is transformed from one of the first and second images is as similar as possible to the other of the first and second images. Preferably, (numerical) optimisation algorithms are applied in order to find the transformation which results in an optimum degree of similarity. The degree of similarity is preferably measured by way of a measure of similarity (also referred to in the following as a “similarity measure”). The parameters of the optimisation algorithm can be in particular vectors of a deformation field. These vectors are determined by the optimisation algorithm in such a way as to result in an optimum degree of similarity. Thus, the optimum degree of similarity represents a condition, in particular a constraint, for the optimisation algorithm. The bases of the vectors lie at voxel positions of one of the first and second images which is to be transformed, and the tips of the vectors lie at the corresponding voxel positions in the transformed image. A plurality of these vectors are preferably provided, for instance more than twenty or a hundred or a thousand or ten thousand, etc. Preferably, there are (other) constraints on the transformation (deformation), in particular in order to avoid pathological deformations (for instance, all the voxels being shifted to the same position by the transformation). These constraints include in particular the constraint that the transformation is regular, which in particular means that a Jacobian determinant calculated from a matrix of the deformation field (in particular, the vector field) is larger than zero, and also the constraint that the transformed (deformed) image is not self-intersecting and in particular that the transformed (deformed) image does not comprise faults and/or ruptures. The constraints include in particular the constraint that if a regular grid is transformed simultaneously with the image and in a corresponding manner, the grid is not allowed to interfold at any of its locations. The optimising problem is can be solved iteratively, in particular by means of an optimisation algorithm which is in particular a first-order optimisation algorithm, in particular a gradient descent algorithm. Other examples of optimisation algorithms include optimisation algorithms which do not use derivations, such as the downhill simplex algorithm, or algorithms which use higher-order derivatives such as Newton-like algorithms. The optimisation algorithm preferably performs a local optimisation. If there are a plurality of local optima, global algorithms such as simulated annealing or generic algorithms can be used. In the case of linear optimisation problems, the simplex method can for instance be used.

In the steps of the optimisation algorithms, the voxels are for example shifted by a magnitude in a direction such that the degree of similarity is increased. This magnitude is preferably less than a predefined limit, for instance less than one tenth or one hundredth or one thousandth of the diameter of the image, and in particular about equal to or less than the distance between neighbouring voxels. Large deformations can be implemented, in particular due to a high number of (iteration) steps.

The determined elastic fusion transformation can be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can be determined on the basis of a determined correlation between the first and second datasets.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements, and optionally a volatile memory (in particular a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which comprises technical, in particular tangible components, in particular mechanical and/or electronic components. Any device mentioned as such in this document is a technical and in particular tangible device.

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The marker is preferably passive, i.e. reflects for example electromagnetic radiation in the infrared, visible and/or ultraviolet spectral range or blocks x-ray radiation. To this end, the marker can be provided with a surface which has corresponding reflective properties or can be made of metal in order to block the x-ray radiation. It is also possible for the marker to reflect electromagnetic radiation and/or waves in the radio frequency range or at ultrasound wavelengths. The marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is in particular known to a navigation system and is for example stored in a computer of the navigation system.

An atlas typically consists of a plurality of generic models of objects, wherein the generic models of the objects together form a complex structure. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

The method in accordance with the invention is in one embodiment a data processing method. The data processing method is preferably performed using technical means, in particular a computer. The data processing method is preferably constituted to be executed by or on a computer and in particular is executed by or on the computer. In particular, all the steps or merely some of the steps (i.e. less than the total number of steps) of the method in accordance with the invention can be executed by a computer. The computer comprises a processor and a memory in order to process the data, in particular electronically and/or optically. The calculating steps described are performed by a computer. Determining steps or calculating steps include steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can comprise a system (network) of “sub-computers”, wherein each sub-computer represents a computer in its own right. The term “computer” includes a cloud computer, in particular a cloud server. The term “cloud computer” includes a cloud computer system which in particular comprises a system of at least one cloud computer and a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. In particular, the term “cloud” is used in this respect as a metaphor for the Internet (world wide web). In particular, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer may comprise interfaces in order to receive or output data and/or perform an analogue-to-digital conversion. The data may in particular be data which represent physical properties and/or which are generated from technical signals. The technical signals are for example by means of (technical) detection devices (such as for example devices for detecting marker devices) and/or (technical) analytical devices (such as for example devices for performing imaging methods), wherein the technical signals are in particular electrical or optical signals. The technical signals may represent the data received or outputted by the computer. The computer is preferably operatively coupled to a display device which allows information outputted by the computer to be displayed, for example to a user. One example of a display device is an augmented reality device (also referred to as augmented reality glasses) which can be used as “goggles” for navigating. A specific example of such augmented reality glasses is Google Glass (a trademark of Google, Inc.). An augmented reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital light box. The monitor could also be the monitor of a portable device, in particular a handheld device such as a smartphone or personal digital assistant or digital media player.

The expression “acquiring data” in particular encompasses (within the framework of a data processing method) the scenario in which the data are determined by the data processing method or program. Determining data in particular encompasses measuring physical quantities and transforming the measured values into data, in particular digital data, and/or computing the data by means of a computer and in particular within the framework of the method in accordance with the invention. The meaning of “acquiring data” also in particular encompasses the scenario in which the data are received or retrieved by the data processing method or program, for example from another program, a previous method step or a data storage medium, in particular for further processing by the data processing method or program. The expression “acquiring data” can therefore also for example mean waiting to receive data and/or receiving the data. The received data can for example be inputted via an interface. The expression “acquiring data” can also mean that the data processing method or program performs steps in order to (actively) receive or retrieve the data from a data source, for instance a data storage medium (such as for example a ROM, RAM, database, hard drive, etc.), or via the interface (for instance, from another computer or a network). The data can be made “ready for use” by performing an additional step before the acquiring step. In accordance with this additional step, the data are generated in order to be acquired. The data can be detected or captured (for example by an analytical device). Alternatively or additionally, the data are inputted in accordance with the additional step, for instance via interfaces. The data generated can in particular be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, in particular determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

In particular, the invention does not involve or in particular comprise or encompass an invasive step which would represent a substantial physical interference with the body requiring professional medical expertise to be carried out and entailing a substantial health risk even when carried out with the required professional care and expertise. In particular, the invention does not comprise a step of positioning a medical implant in order to insert it into a patient's body or a step of inserting the medical implant into the patient's body or a step of preparing the patient's body for having the medical implant inserted into it. More particularly, the invention does not involve or in particular comprise or encompass any surgical or therapeutic activity. The invention is instead directed as applicable to determining the structure of a medical implant, which may be outside the patient's body. For this reason alone, no surgical or therapeutic activity and in particular no surgical or therapeutic step is necessitated or implied by carrying out the invention.

It is within the scope of the present invention to combine one or more features of one or more embodiments in order to form a new embodiment wherever this is technically expedient and/or feasible. Specifically, a feature of one embodiment which has the same or a similar function to another feature of another embodiment can be exchanged for said other feature, and a feature of one embodiment which adds an additional function to another embodiment can in particular be added to said other embodiment.

Such a method can be applied in combination with the auto-segmentation portfolio of the Applicant, which serves as a background segmentation process generating planning objects on the basis of a patient's current anatomy. Such a cavity-filling object can automatically be calculated by the system and be presented to the user, either for export via e.g. an STL interface for printing or for other purposes. Furthermore, the combination of such a system with manual editing software (such as e.g. the Smartbrush) for further modification of the object and the application with an IGS station for optimal placement of the object can enhance the surgical workflow in terms of time saving, safety and overall efficiency.

In the following, the invention is described with reference to the enclosed figures which represent preferred embodiments of the invention. The scope of the invention is not however limited to the specific features disclosed in the figures, which show:

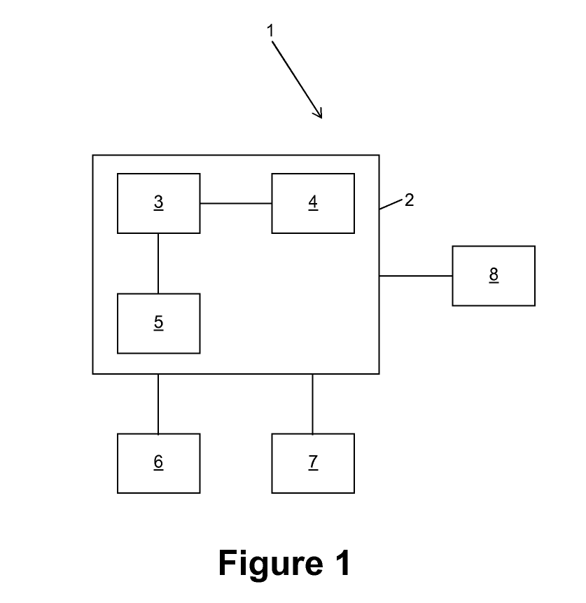

FIG. 1 a system according to the present invention;

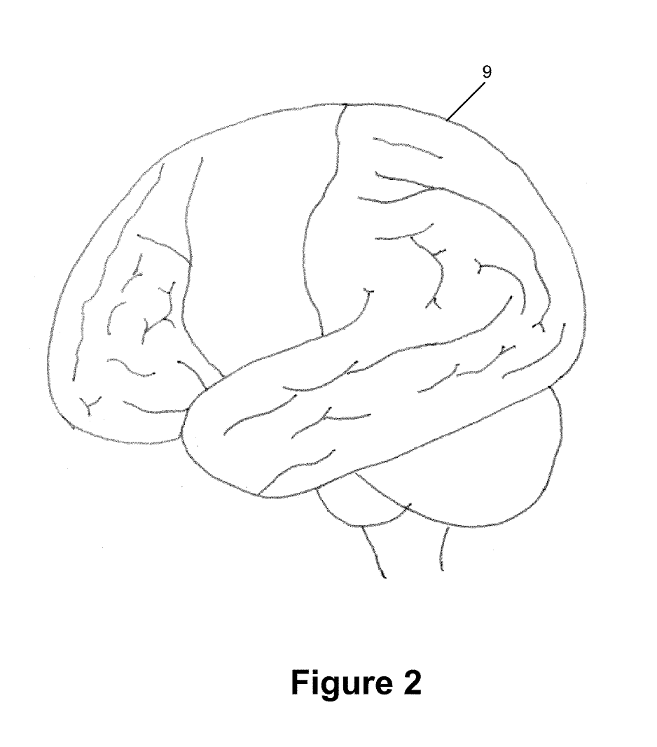

FIG. 2 an illustration of a brain in a pre-operative state;

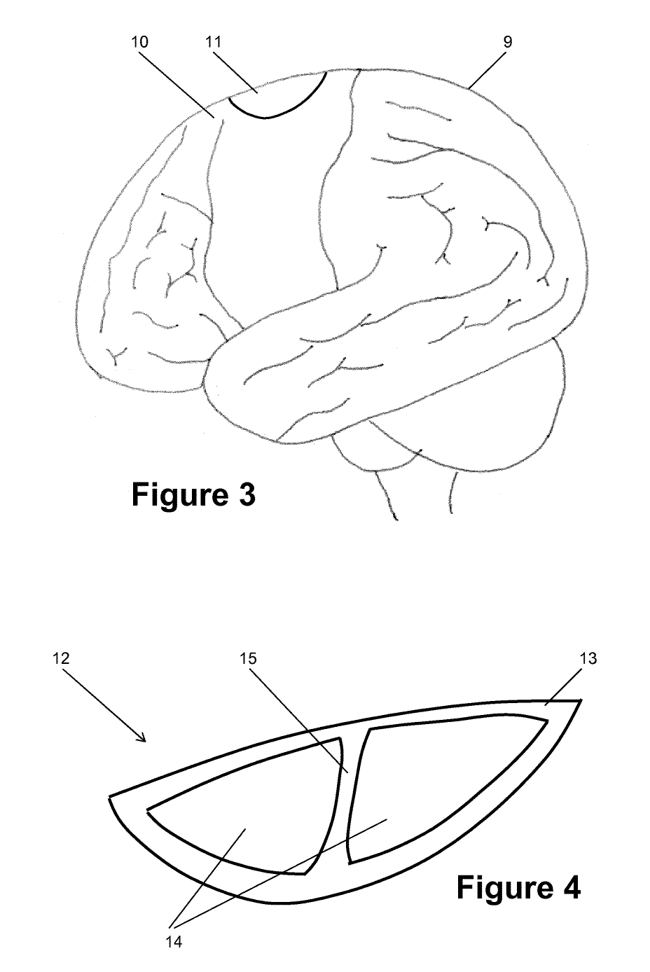

FIG. 3 an illustration of the brain from FIG. 2 after a first type of resection;

FIG. 4 an implant for replacing the removed tissue in the brain as shown in FIG. 3;

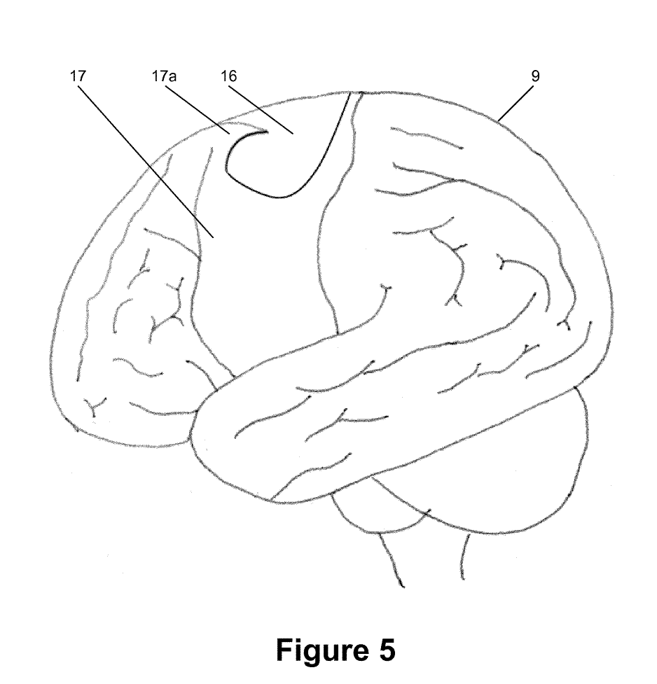

FIG. 5 an illustration of the brain from FIG. 2 after a second type of resection;

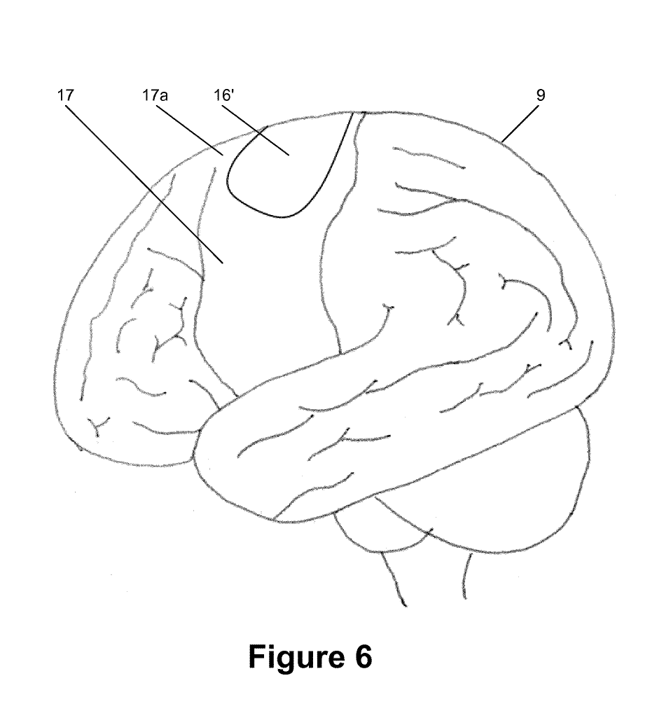

FIG. 6 the illustration of the brain from FIG. 5 after it has been matched to the illustration of the brain from FIG. 2;

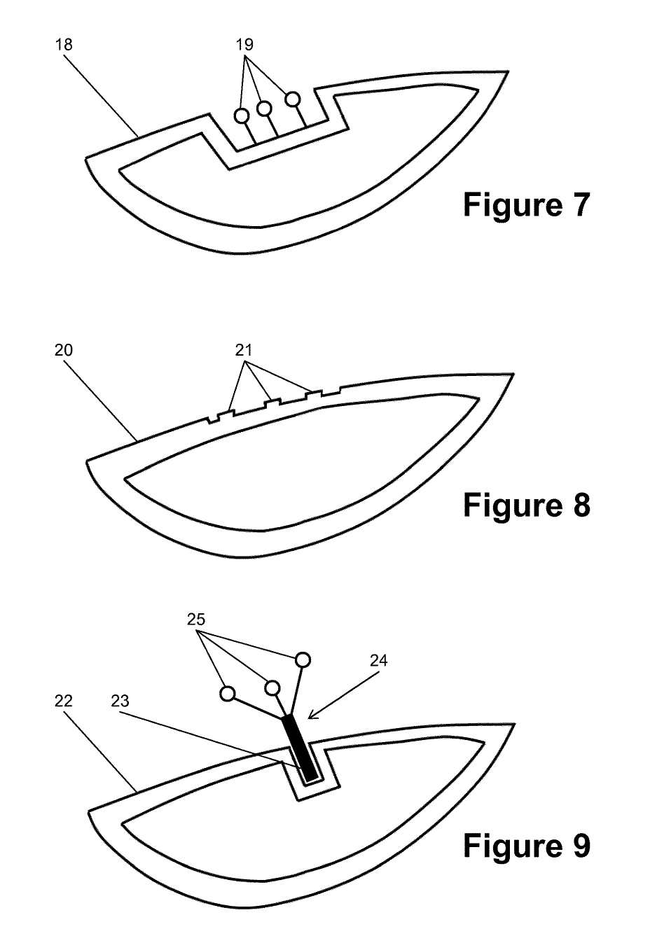

FIG. 7 an implant comprising marker spheres;

FIG. 8 an implant comprising embossed marker areas;

FIG. 9 an implant comprising a socket for a marker device;

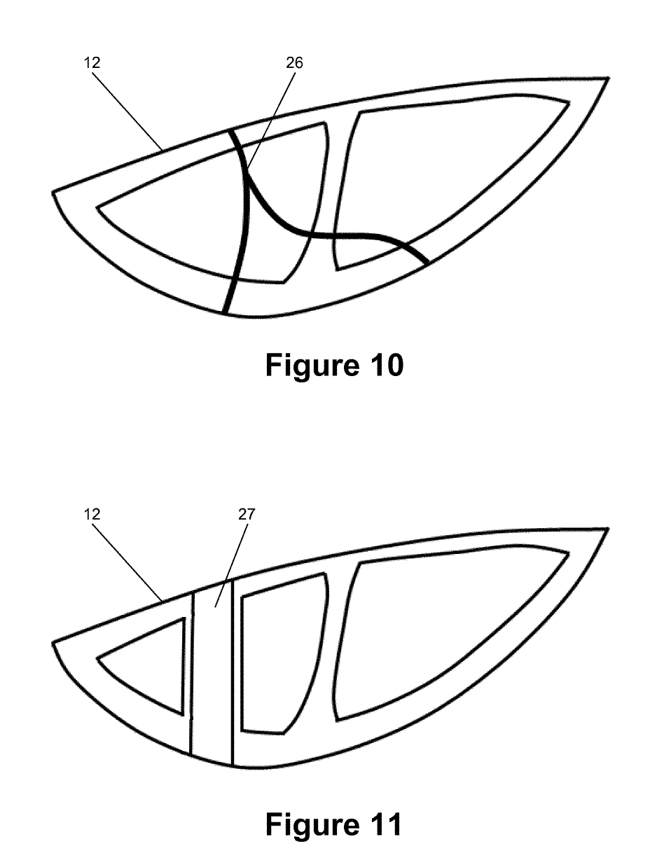

FIG. 10 an implant comprising a conduit; and

FIG. 11 an implant comprising a port.

FIG. 1 shows a system 1 for determining the structure of a medical implant which is to replace removed tissue in a patient's body. The system 1 comprises a computer 2 which in turn comprises a central processing unit 3, a memory 4 and an interface 5. The computer 2 is connected to an input unit 6, such as a keyboard, a mouse or a touch-sensitive surface, and an output unit 7 such as a monitor. The computer 2 is also connected to a medical imaging device 8 which provides one or more 3D datasets. The medical imaging device 8 can be replaced by any device which is capable of providing a 3D dataset, such as a storage medium, a server or another computer. Via the interface 5, the computer 2 exchanges data with devices connected to the computer 2. The central processing unit 3 performs the method steps as described in this document. The memory 4 stores program code which instructs the central processing unit 3 to perform the method and acts as a working memory for data which have been or are to be processed by the central processing unit 3.

FIG. 2 shows a brain 9 in a pre-operative state in which the brain 9 comprises tissue to be removed by cranial resection and tissue which is to remain after the resection. An initial 3D dataset which represents the brain 9 in the state shown in FIG. 2 is recorded by the medical imaging device 8 and acquired by the computer 2 from the medical imaging device 8.

FIG. 3 shows the brain 9 from FIG. 2 after a cranial resection. Parts of the brain tissue have been removed, leaving an area of remaining tissue 10 and a volume 11 which contained the removed tissue before the resection was performed. The medical imaging device 8 creates a first medical imaging dataset which represents the brain 9 in the state shown in FIG. 3. The computer 2 acquires the first medical image dataset from the medical imaging device 8.

The central processing unit 3 analyses the first medical imaging dataset and identifies the shape and size of the volume 11. In order to prevent damage to the remaining tissue 10, the volume 11 is to be filled with a medical implant. The central processing unit 3 determines the required contour of the implant from the shape and the size of the volume 11.

The central processing unit 3 then simulates the forces which would be exerted by the remaining tissue 10 on the contour of the implant if the implant were situated in the volume 11. For this purpose, the central processing unit 3 assumes the maximum accelerations which could act on the brain 9 and determines the forces exerted by the remaining tissue 10 which surrounds the volume 11 on the basis of these maximum accelerations and the structure of the remaining tissue 10. This simulation can for example be based on a finite element approach or any other suitable simulation technique, such as a technique based on a mass-spring model.

One option for simulating the forces exerted by the remaining tissue is to use an atlas of the brain which is matched to the patient's brain 9, in order to segment the remaining tissue 10 into tissue types. The simulation can then be based on the behaviour of the remaining tissue of each respective tissue type, for example by setting the masses and spring properties in the mass-spring model accordingly.

The central processing unit 3 then determines a structure dataset which represents the structure of the implant such that the implant has the determined required contour and can absorb the simulated forces. A sectional view of an example implant 12 is shown in FIG. 4. The contour of the implant 12 completely fills the volume 11 within the brain 9. In the present example, the medical implant 12 is a hollow implant in which an implant wall 13 encloses a hollow volume 14. The medical implant 12 also comprises an example of a stiffener 15 which connects opposite parts of the implant wall 13 in order to increase the structural stability of the implant.

The stiffener 15 and the thickness of the walls 13 are configured such that the medical implant 12 has a rigidity which matches the rigidity of the removed tissue. From a mechanical point of view, the implant 12 thus behaves like the removed tissue. The rigidity of the implant depends on a number of parameters, such as the thickness of the implant walls, the structure of any stiffeners within the implant, and the presence or absence of (micro-)cavities in the implant walls and/or stiffener(s).

It should be noted that the implant 12 need not necessarily comprise a sealed hollow volume 14. The implant 12 can also for example be permeable to a cerebrospinal fluid. The implant 12 can optionally also comprise conduits for a medical liquid, such as for example a system of conduits comprising one inlet port through which the medical liquid is introduced and a plurality of outlet ports through which the introduced medical liquid is provided to the remaining tissue 10.

The medical implant 12 can optionally also include at least one port through which a medical instrument can pass, such that the medical instrument can reach the remaining tissue 10 through the implant 12.

In the first example described above, the contour of the implant 12 is derived directly from the first medical image dataset. Depending on the type of resection, this may not result in the correct, i.e. required contour for the implant. FIG. 5 shows the brain 9 in its state after a second type of cranial resection, comprising a volume 16 and remaining tissue 17. However, the shape of the volume 16 does not match the shape of the removed tissue because some of the remaining tissue 17 has moved due to the force of gravity after the resection was performed, as indicated by the reference numeral 17a in FIG. 5. The contour of the implant should however match the shape of the removed tissue rather than the shape of the volume 16 in FIG. 5.

In this case, the medical imaging device 8 generates a medical imaging dataset which represents the brain 9 after the cranial resection, as in the first example. In this second example, however, the medical imaging dataset is referred to as the second medical imaging dataset and represents the state of the brain 9 as shown in FIG. 5.

Before the central processing unit 3 determines the required contour of the implant, it matches the second medical imaging dataset, which represents the state of the brain 9 as shown in FIG. 5, to the initial medical imaging dataset which represents the state of the brain 9 as shown in FIG. 2. The result of this matching process is a matched second medical imaging dataset which then represents a matched brain state, as shown in FIG. 6. The result of the matching step is that the position of the remaining tissue 17a in the matched second medical imaging dataset is the same as its position in the initial medical imaging dataset. The volume 16 in the second medical imaging dataset is thus transformed into the volume 16′ in the matched second medical imaging dataset, which has the shape of the removed tissue. The central processing unit 3 then determines the required contour of the implant from the matched second medical imaging dataset.

The structure of the implant generally consists of the contour of the implant and the internal arrangement of the implant. In one example, the structure dataset is a three-dimensional array of binary information indicating whether or not material is present at a position defined by the position of the binary information within the array. Printing data can also for example be generated from the structure of the implant, such that the implant can be produced using a 3D printer.

FIG. 7 shows an example implant 18 which comprises three marker spheres 19 within its structure. The marker spheres 19 are preferably formed integral with the implant 18. The marker spheres 19 have a known size and are arranged in a known spatial relationship, such that the position of the implant 18 in space can be determined from the locations of the marker spheres 19 in space. The spatial location of the marker spheres 19 is for example obtained via a medical navigation system. The marker spheres 19 are preferably reflective for infrared light. They can be provided with an IR-reflective coating or be made of an IR-reflective material, for example by using multiple materials in the 3D printing process of the implant 18.

FIG. 8 shows another example of an implant 20 which comprises embossed areas 21. The sizes and spatial arrangement of the areas 21 is known, such that the spatial position of the implant 20 can be calculated from the spatial locations of the areas 21. The areas 21 are preferably circular. They also preferably reflect infrared light, for example by being provided with an IR-reflective coating or made from an IR-reflective material.

FIG. 9 shows another example of a medical implant 22 which comprises a socket 23 for receiving a marker device 24. The marker device 24 comprises at least three marker spheres 25 in a known spatial arrangement. If the marker device 24 is plugged into the socket 23, the spatial position of the implant 22 can be calculated from the spatial locations of the marker spheres 25. The marker spheres 25, like the marker spheres 19 of FIG. 7, reflect infrared light.

FIG. 10 shows the implant 12 with an additional conduit 26 which has an inlet portion and two outlet portions. A medical liquid can be administered through the conduit 26 by introducing it into the inlet portion, from where it is guided to the outlet portions close to the remaining tissue.

FIG. 11 shows the medical implant 12 with a port 27 which connects opposite sides of the implant 12. The size of the port 27 is selected such that a medical instrument can be guided through the port 27, and therefore through the implant 12, in order to reach the remaining tissue without the implant 12 having to be removed.

1-15. (canceled)

16. A system for determining the structure of a medical implant which is to replace a removed tissue in a patient's body, comprising a computer having a processor which performs the following steps:
acquiring a 3D dataset which represents a remaining tissue which at least partly surrounded the removed tissue before removal;
determining a required contour of the medical implant from the 3D dataset;
identifying a simulated force exerted by the remaining tissue on a contour of the medical implant; and
determining a structure dataset which represents a structure of the medical implant such that the medical implant has the required contour to absorb the simulated force;
wherein the step of determining the structure dataset involves configuring a wall thickness of the medical implant that is capable of withstanding at least the simulated force.

17. A data processing method, performed by a processor of a computer, for determining a structure of a medical implant which is to replace a removed tissue in a patient's body, comprising the steps of:
acquiring, by the processor, a 3D dataset which represents a remaining tissue which at least partly surrounded the removed tissue before the latter was removed;
determining, by the processor, a required contour of the medical implant from the 3D dataset;
simulating, by the processor, forces exerted by the remaining tissue on a contour of the medical implant; and
determining, by the processor, a structure dataset which represents a structure of the medical implant such that the medical implant has the required contour and can absorb the simulated forces;
wherein the step of determining the structure dataset involves configuring, by the processor, a wall thickness of the medical implant.

18. The method according to claim 17, wherein the step of determining the structure dataset involves providing, by the processor, the structure with at least one stiffener.

19. The method according to claim 17, wherein the 3D dataset is a medical imaging dataset.

20. The method according to claim 17, wherein the step of determining the required contour of the medical implant from the 3D dataset involves determining, by the processor, a shape of a cavity formed by the remaining tissue in the 3D dataset and using, by the processor, the shape of the cavity as the contour of the medical implant.

21. The method according to claim 17, wherein the step of determining the required contour of the medical implant from the 3D dataset involves matching, by the processor, the 3D dataset to an initial 3D dataset which represents at least parts of the remaining tissue before the removed tissue was removed, in order to determine a shape of a cavity formed by the remaining tissue in the matched 3D dataset, and using, by the processor, the shape of the cavity as the contour of the medical implant.

22. The method according to claim 21, wherein the initial 3D dataset is a medical imaging dataset or a matched atlas.

23. The method according to claim 17, wherein the step of simulating forces exerted by the remaining tissue involves segmenting, by the processor, the remaining tissue on a basis of an atlas and simulating a movement of a segmented remaining tissue.

24. The method according to claim 17, wherein the step of determining the structure dataset involves providing, by the processor, the medical implant with a rigidity which reflects the rigidity of the removed tissue.

25. The method according to claim 17, wherein the step of determining the structure dataset involves arranging, by the processor, the structure of the medical implant so as to form at least one marker which is to be detected by a medical navigation system.

26. The method according to claim 17, wherein the step of determining the structure dataset involves providing, by the processor, the structure of the medical implant with a socket for a marker device which is to be detected by a medical navigation system.

27. The method according to claim 17, wherein the step of determining the structure dataset involves providing, by the processor, the structure of the medical implant with at least one port for a medical liquid or at least one conduit for a medical liquid.

28. A method for producing a medical implant structure, comprising performing the method of claim 17 and further comprising the steps of producing the medical implant structure as represented by the structure dataset and verifying the contour of the produced implant.

29. A non-transitory computer-readable storage medium storing a computer program which, when running on a computer, causes the computer to perform the following steps:
acquiring a 3D dataset which represents a remaining tissue which at least partly surrounded a removed tissue before removal;
determining a required contour of an implant from the 3D dataset to replace the removed tissue in a patient's body;
performing a simulation of forces exerted by the remaining tissue on a contour of the implant; and
determining a structure dataset which represents a structure of the implant such that the implant has the required contour;
wherein the step of determining the structure dataset involves configuring a wall thickness of the implant.

30. A computer comprising the non-transitory storage medium of claim 29.

31. The system of claim 16, further comprising the processor identifying a shape of a cavity formed by the remaining tissue in the 3D dataset and using the shape identified as the contour of the medical implant.

32. The system of claim 16, wherein the structure of the medical implant includes at least one stiffener.

33. The system of claim 16, further comprising:
the processor utilizing an initial 3D dataset representing at least parts of the remaining tissue before removal; and
the processor matching the 3D dataset to the initial 3D dataset to identify a shape of a cavity formed by the remaining tissue and using the shape identified as the required contour of the medical implant.

34. The system of claim 16, wherein the structure of the medical implant includes a rigidity which reflects the rigidity of the removed tissue or withstands the simulated force.

35. The system of claim 16, wherein the structure of the medical implant includes at least one marker which is to be detected by a medical navigation system.
